(19) United States
(12) Patent Application Publication
(10) Pub. No.: US 2025/0302450 A1
(43) Pub. Date: Oct. 2, 2025

(54) HYDROSOL, ULTRASOUND PHANTOM USING THE HYDROSOL, ULTRASOUND PHANTOM SET, AND METHOD FOR MANUFACTURING ULTRASOUND PHANTOM

(71) Applicant: CANON KABUSHIKI KAISHA, Tokyo (JP)

(72) Inventors: CHIAKI NISHIURA, Tokyo (JP); KYOHEI WADA, Tokyo (JP); RYO OGAWA, Kanagawa (JP)

(21) Appl. No.: 19/090,600

(22) Filed: Mar. 26, 2025

(30) Foreign Application Priority Data
Mar. 27, 2024 (JP) 2024-051949
Feb. 14, 2025 (JP) 2025-022738

(51) Int. Cl. A61B 8/00; G09B 23/28

(52) U.S. Cl. CPC A61B 8/587; G09B 23/286

(57) ABSTRACT

The present disclosure provides a hydrosol in which a velocity of sound is close to a velocity of sound in a living body and viscosity is high. In order to solve the problem, the present disclosure provides a hydrosol that is applied to an ultrasound phantom, and includes water, cellulose ether and a sound velocity adjusting agent.

### BACKGROUND OF THE INVENTION

### Field of the Invention

The present disclosure relates to a hydrosol, an ultrasound phantom using the hydrosol, an ultrasound phantom set, and a method for manufacturing the ultrasound phantom.

### Description of the Related Art

Medical image diagnostic equipment such as an acoustic wave diagnostic apparatus, a magnetic resonance imaging diagnostic apparatus, an X-ray image diagnostic apparatus and a near-infrared light imaging apparatus perform diagnosis by irradiating a site to be observed with a sound wave, an electromagnetic wave, etc., and observing reflected or transmitted sound waves and electromagnetic waves. A living tissue simulating material (hereinafter referred to as phantom) which simulates reflection/absorption characteristics of sound waves, electromagnetic waves, etc. of a living body is used for calibration of the above image diagnostic apparatus and for expansion of a measurement range.

An ultrasound diagnostic apparatus is an apparatus that observes a reflected wave of an ultrasound that has been emitted to an observation site and visualizes an internal state. In recent years, various analysis applications have been developed and installed. For example, the applications are a shear wave elastography method which can quantify the Young's modulus by measurement of the propagation velocity distribution of shear waves that have been generated by excitation of the observation site; a shear wave dispersion method which can quantify viscosity by measurement of the frequency dependence of the propagation velocity of the generated shear waves; and the like.

It has been enabled to evaluate a progress of a pathology, which has been difficult to be distinguished by a single evaluation method, by combination of a plurality of the measurement methods described in the examples. For example, a method has been proposed which combines the aforementioned shear wave elastography method and the shear wave dispersion method, as a new approach for understanding the pathology of a liver.

On the other hand, in order to correctly evaluate a progress of pathology by an ultrasound diagnostic apparatus, it is necessary to calibrate the apparatus with a use of an ultrasound phantom of which the Young's modulus, viscosity, etc. are obvious. As a method for producing a phantom of which viscosity is controlled, a method of adding glycerin which is a viscous liquid to a phantom is disclosed in Japanese Patent No. 6754112.

In general, a hydrogel which is composed of water as a main component is particularly suitable as a phantom for an ultrasound diagnostic apparatus, in other words, an ultrasound phantom, because velocity of sound of longitudinal wave is close to that of a living body and acoustic attenuation is small. When a hydrogel is used as an ultrasound phantom, a substance for adjusting the velocity of sound of the longitudinal wave is generally added to the hydrogel, in order to bring the velocity of sound of the longitudinal wave close to that of a living body.

On the other hand, when a substance is added for a purpose of adjusting physical properties of a hydrogel other than the velocity of sound of the longitudinal wave, an unintended change in the velocity of sound of the longitudinal wave occurs. For example, in a hydrogel disclosed in Japanese Patent No. 6754112, a large amount of glycerin is added for a purpose of enhancing viscosity, and thereby an enhancement of viscosity of the hydrogel is achieved. However, when the hydrogel is used as an ultrasound phantom, it is a problem that the velocity of sound of the longitudinal wave results in being far away from that of the living body.

Therefore, an object of the present disclosure is to provide a hydrosol in which a velocity of sound is close to the velocity of sound in a living body and viscosity is high. In addition, an object of the present disclosure is to provide an ultrasound phantom by use of a hydrosol in which a velocity of sound is close to the velocity of sound in a living body and viscosity is high. In addition, an object of the present disclosure is to provide an ultrasound phantom set with the use of a hydrosol in which a velocity of sound is close to the velocity of sound in a living body and viscosity is high. In addition, an object of the present disclosure is to provide a method for manufacturing an ultrasound phantom by use of a hydrosol in which a velocity of sound is close to the velocity of sound in a living body and viscosity is high.

### SUMMARY OF THE INVENTION

The present disclosure provides a hydrosol that is applied to an ultrasound phantom, and includes water, cellulose ether, and a sound velocity adjusting agent. In addition, the present disclosure provides an ultrasound phantom that includes the above hydrosol, and a container which has a retaining portion that is in contact with the hydrosol and retains a shape of the hydrosol, and accommodates the hydrosol. In addition, the present disclosure provides an ultrasound phantom set that includes the ultrasound phantom described above; and a second ultrasound phantom that includes a second cellulose ether having an average molecular weight different from the average molecular weight of the cellulose ether contained in the ultrasound phantom, water, a sound velocity adjusting agent, and a second container that accommodates a second cellulose ether, the water, and a sound velocity adjusting agent. In addition, the present disclosure provides a method for manufacturing the ultrasound phantom that includes: producing a mixed liquid in which water, cellulose ether and a sound velocity adjusting agent are mixed; producing a hydrosol by stirring and mixing the mixed liquid; and pouring the hydrosol into a container.

Further features of the present disclosure will become apparent from the following description of exemplary embodiments.

### DESCRIPTION OF THE EMBODIMENTS

Preferred embodiments of the present disclosure will now be described in detail.

In the present disclosure, the description of “XX or more and YY or less” or “XX to YY” which represents a numerical range means a numerical range that includes a lower limit and an upper limit which are end points, unless otherwise specified. When numerical ranges are listed in a stepwise manner, upper and lower limits of each numerical range can be arbitrarily combined.

### First Embodiment

The first embodiment is about a hydrosol.

The hydrosol of the present embodiment is a hydrosol which is applied to the ultrasound phantom, and includes water, cellulose ether and a sound velocity adjusting agent.

The hydrosol applied to the ultrasound phantom according to the present embodiment, and the materials and the configuration contained in the ultrasound phantom which uses the hydrosol will be described below.

### <Description of Hydrosol>

The hydrosol of the present embodiment is a hydrosol which is applied to an ultrasound phantom. The hydrosol in the present embodiment means a sol that contains water as a main component and has fluidity; and is a sol that contains water and a sound velocity adjusting agent which will be described later, where at least a part of cellulose ether which will be described later dissolves in water. If necessary, the sol may be mixed with other components which will be described later and may be accommodated in a container which will be described later, and thereby can be used as an ultrasound phantom.

### <Description of Water>

The hydrosol of the present embodiment contains water. As the water which is used in the present embodiment, tap water, well water, pure water, ultrapure water or the like can be used. Purified water is preferable from a viewpoint of suppressing a fluctuation of physical properties due to impurities. Examples of methods for purifying water include: distillation; methods of using a reverse osmosis membrane, an ion exchange membrane, or sterilization with an use of a UV lamp; and methods combining these methods.

In the hydrosol, it is preferable that at least a part of water is hydrated with cellulose ether which will be described later. A water content in the hydrosol is, for example, preferably 50.00 mass % or more and 99.00 mass % or less, more preferably 70.00 mass % or more and 99.00 mass % or less, further preferably 80.00 mass % or more and 99.00 mass % or less, and further more preferably 84.00 mass % or more and 94.00 mass % or less.

### <Description of Cellulose Ether>

The hydrosol of the present embodiment contains cellulose ether. The cellulose ether in the present embodiment is a chemical compound of which the basic skeleton is a polysaccharide in which a large number of glucoses that are monosaccharides are linearly linked, and in which a part of hydroxy groups of the glucose is substituted with an alkoxy group.

A bond which connects glucoses to each other is preferably a glycosidic bond from the viewpoint of availability. In addition, it is preferable that three-dimensional conformation of the glucose is D-form, from a viewpoint of availability, but may be L-form, or may be the mixture. Furthermore, the cellulose ether may partially contain a glucose in which a hydroxy group bonded to an anomeric carbon is a cis-form (α,-anomer). From a viewpoint of availability, the cellulose is preferable in which the basic skeleton includes 90% or more of β-D-glucose. Hereinafter, glucose after having been bonded by a glycosidic bond is referred to as a glucose residue.

The alkoxy group which substitutes a part of hydroxy groups of glucose residue may be linear or branched, and may contain a hydroxy group. Furthermore, a hydroxy group of the alkoxy group containing the hydroxy group may be further substituted with an alkoxy group. Hereinafter, the glucose residue is referred to as a glucose residue, regardless of whether the hydroxy group of the glucose residue is substituted with an alkoxy group or not.

### <Types and Substituents of Cellulose Ethers>

Examples of the cellulose ethers which are suitably used in the present embodiment include carboxymethyl cellulose, methyl cellulose, ethyl cellulose, hydroxyethyl cellulose, hydroxyethyl methyl cellulose, hydroxypropyl cellulose, and hydroxypropyl methyl cellulose.

In the cellulose ether of the present embodiment, it is preferable that a hydroxy group derived from the glucose residue is substituted with at least one selected from the group consisting of a methoxy group, a hydroxyethoxy group and a hydroxypropoxy group. It is preferable for the alkoxy group substituting a part of the hydroxy groups of the glucose residue to be a methoxy group, an ethoxy group, a hydroxyethoxy group, a hydroxypropoxy group, or a carboxymethoxy group, from a viewpoint of availability, and is more preferable to be the methoxy group, the hydroxyethoxy group or the hydroxypropoxy group, from a viewpoint of water solubility suitable for the production of a hydrosol.

It is preferable in the cellulose ether of the present embodiment that one glucose residue is modified with a methoxy group at a proportion of 0.4 or larger and 2.4 or smaller, with a hydroxyethoxy group at a proportion of 0 or larger and 0.6 or smaller, and with a hydroxypropoxy group at a proportion of 0 or larger and 0.5 or smaller, on average.

As for an average number of the alkoxy groups contained in one glucose residue, it is preferable for methoxy groups to be 0.4 to 2.4 groups, and is more preferably to be 0.9 to 1.9 groups, from a viewpoint of availability and water solubility suitable for a production of a hydrosol. It is preferable for hydroxyethoxy groups to be 0 to 0.6 groups, and is more preferably to be 0 to 0.4 groups. It is preferable for hydroxypropoxy groups to be 0 to 0.5 groups, and is more preferably to be 0.1 to 0.3 groups.

In addition, when an alkoxy group containing a hydroxy group is introduced as a substituent of a glucose residue of cellulose ether, the hydroxy group may be also substituted with an alkoxy group, and accordingly, alkoxy groups may be added more than 3 which is a number of hydroxy groups originally possessed by the glucose residue.

### <Surface Treatment of Cellulose Ether>

It is preferable that the cellulose ether in the present embodiment is subjected to surface treatment with a hydrophobic substance, for the purpose of enhancement of dispersibility in water.

It is preferable that hydrophobic substance used in the surface treatment is a substance which gradually causes a decomposition reaction in water and becomes water-soluble, and is glyoxal from a viewpoint of availability.

### <Content of Cellulose Ether>

In the hydrosol according to the present embodiment, it is preferable for purity of the cellulose ether to be 80 mass % or more, is more preferable to be 90 mass % or more, and is further preferable to be 95 mass % or more. In the hydrosol according to the present embodiment, it is preferable that the purity of the cellulose ether is 98 mass % or less.

It is preferable for a content of the cellulose ether to be 1.5 parts by mass or more, and is preferable to be 1.5 parts by mass or more and 5.0 parts by mass or less in order to obtain a uniform hydrosol that ensures defoaming properties. It is more preferable for the content to be 1.5 parts by mass or more and 4.0 parts by mass or less, and is further preferable to be 2.0 parts by mass or more and 4.0 parts by mass or less.

In the hydrosol of the present embodiment, it is preferable that a ratio (mass ratio) Cc/Cw of the content of cellulose ether to the content of water is 1.5/98 or larger and 5/80 or smaller, in other words, 1.53×10⁻² or larger and 6.25×10⁻² or smaller.

### <Thickening Mechanism of Cellulose Ether>

The cellulose ether which is used in the hydrosol of the present embodiment dissolves in water at room temperature (25° C.) to form the hydrosol. In a state of hydrosol, when a force is applied, resistance is generated due to entanglement of polymer chains contained in the cellulose ether. A strength of the entanglement of the polymer chains can be adjusted by an amount and molecular weight of the cellulose ether. The molecular weight referred to here refers to a number average molecular weight or a mass average molecular weight.

For example, when cellulose ether having a large molecular weight is used, an entanglement of the polymers is strong, and accordingly, a network between molecules is easily formed; and even a small amount of addition strengthens elastic properties in viscoelasticity. On the other hand, when a cellulose ester having a low molecular weight is used, the entanglement is weak, and accordingly, the elastic properties are not easily strengthened even though an additive amount is large.

Accordingly, the elasticity and viscosity in the viscoelasticity can be arbitrarily controlled by changing the molecular weight and additive amount of the cellulose ether to be used.

Due to the above mechanism of thickening, the cellulose ether can be suitably used for simulating the viscosity of organs in particular.

### <Properties of Cellulose Ether Containing Methoxy Group>

Among the cellulose ethers in the hydrosol of the present embodiment, the hydrosol containing a cellulose ether containing a methoxy group is a hydrosol at room temperature, but undergoes phase transition to a hydrogel by heating, and this phase transition is reversible. This is considered to be because the interaction of the hydrophobic groups is strengthened by thermal motion, when a temperature becomes high.

A phase transition temperature varies depending on the number of methoxy groups and the type of other alkoxy groups, but has a transition point between 65° C. and 90° C. It can be determined whether the cellulose ether is in a state of hydrosol or hydrogel, by measurement of viscoelasticity, which will be described later.

### <Description of Sound Velocity Adjusting Agent>

The hydrosol of the present embodiment contains a sound velocity adjusting agent. The sound velocity adjusting agent which is used in the hydrosol of the present embodiment refers to a material that exhibits a velocity of sound of a longitudinal wave different from that in water alone, when water and the sound velocity adjusting agent have been mixed.

The sound velocity adjusting agent may be in a state of being dissolved in water or in a state of being dispersed in water, but is preferably dissolved in water, from a viewpoint of transmittance of ultrasound. Furthermore, in order to prevent a change in velocity of sound of the longitudinal wave, the sound velocity adjusting agent is preferably an agent which is not easily volatilized, and is preferably an adjusting agent which is not decomposed in water, or does not react with other components.

### <Type of Sound Velocity Adjusting Agent>

Examples of the sound velocity adjusting agent which is used in the hydrosol of the present embodiment include: inorganic compounds such as sodium hydrogen carbonate; organic compounds such as urea, guanidine, guanidine salts, glucose and inositol; alcohols such as ethanol, ethylene glycol and glycerin; and organic solvents such as N,N-dimethyl sulfoxide and N,N-dimethylformamide.

### <Additive Amount of Sound Velocity Adjusting Agent>

The velocity of sound in an organ of a living body is about 1535 m/s, and accordingly, an amount of the sound velocity adjusting agent which is used in the hydrosol of the present embodiment may be added within a range that does not deviate significantly from the value.

For example, it is preferable for the velocity of sound of the ultrasound when an ultrasound having a frequency of 3.5 MHz has transmitted through the hydrosol to be 1500 m/s or larger and 1600 m/s or smaller, is more preferable to be 1520 m/s or larger and 1550 m/s or smaller, and is further preferable to be 1530 m/s or larger and 1540 m/s or smaller.

It is preferable that an additive amount of the sound velocity adjusting agent is 0.1 parts by mass or more with respect to 100.0 parts by mass of water, from a viewpoint of stabilizing the velocity of sound of the longitudinal wave, and is 25 parts by mass or less, from a viewpoint of solubility and dispersibility in water. It is more preferable that the additive amount is 0.5 parts by mass or more and 20 parts by mass or less.

For example, in a case of urea, when mass of water is set to 94.0 parts by mass, it is preferable for an additive amount of urea to be 2.0 parts by mass or more and 10.0 parts by mass or less, is further preferable to be 4.0 parts by mass or more and 8.0 parts by mass or less, and is more preferable to be 5.0 parts by mass or more and 7.0 parts by mass or less.

### <Other Components>

Various components can be added to the hydrosol of the present embodiment as necessary, in addition to water, cellulose ether, and other components different from the sound velocity adjusting agent.

### <Description of Ultrasound Scattering Agent>

The hydrosol of the present embodiment may further contain an ultrasound scattering agent as another component. In the ultrasound diagnostic apparatus, imaging and measurement are performed with a use of a signal that reaches a detector, among ultrasounds scattered in the hydrosol. For this reason, imaging can be performed by addition of an ultrasound scattering agent to a portion to be measured, and thereby a Young's modulus and a viscosity can be measured.

In addition, a scattering efficiency of the ultrasound is calculated by an acoustic impedance (=density×velocity of sound) of the substance. The scattering efficiency at a substance interface increases as a difference in an acoustic impedance value between substances constituting an interface increases.

### <Type of Ultrasound Scattering Agent>

The ultrasound scattering agent may be any of a gas, a liquid and a solid, as long as a difference from water in an acoustic impedance is large which is a main component of the hydrosol. It is preferable for the ultrasound scattering agent to be a liquid or a solid, from a viewpoint of dispersion stability in the hydrosol, and is preferable to be a solid, from a viewpoint of a particle size and mechanical stability.

Examples of a substance that can be used as the ultrasound scattering agent include known substances such as inorganic particles, metals, metal oxides, carbon particles and spherical polymers. Specifically, preferable substances include: particles of carbon crystals such as graphite and microdiamond; particles of amorphous carbon such as carbon black; particles made of resins such as polyethylene particles, polyethylene hollow spheres, and polystyrene hollow spheres; fine particles of oxides such as titanium oxide, alumina oxide and silicon oxide; and fine particles of metals such as tungsten, nickel and molybdenum. Among the substances, particles of carbon crystals are particularly preferable in view of high acoustic impedance and dispersibility in water.

### <Particle Size of Ultrasound Scattering Agent>

A particle size of the ultrasound scattering agent is determined according to a wavelength of the ultrasound to be input. It is preferable that the particle size of the ultrasound scattering agent is 5 μm or larger and 50 μm or smaller, when being calculated from a wavelength of ultrasound emitted from a probe of an ultrasound diagnostic apparatus. The particle size is more preferably 5 μm or larger and 25 μm or smaller.

However, particles having a particle size of 5 μm or larger and a high density generally have a high sedimentation rate, and the particles have a case where the particles of the ultrasound scattering agent may be separated during a thickening of a cellulose ether mixture. A sedimentation rate can be calculated from the following Stokes equation.

$$V = g ( {ρ}_{p}^{-} {ρ}_{f} ) {d}_{p}^{2} / 18 η$$

V: sedimentation rate (m/s), g: gravity acceleration (m/s²), ρp: particle density (kg/m³), ρ_{f}: fluid density (kg/m³), dₚ: particle size (m), η: fluid viscosity (Pa·s)

Accordingly, the sedimentation of the particles of the ultrasound scattering agent can be prevented, by reduction of the particle size of the ultrasound scattering agent, reduction of the difference in density between the ultrasound scattering agent and the fluid, or enhancement of the viscosity of the fluid.

### <Additive Amount of Ultrasound Scattering Agent>

A content of the ultrasound scattering agent may be appropriately adjusted according to a target scattering effect and is not particularly limited; but when mass of water is set to 94.0 parts by mass, it is preferable for a content to be 0.1 parts by mass or more and 30.0 parts by mass or less, is more preferable to be 0.5 parts by mass or more and 20.0 parts by mass or less, and is more preferable to be 3.0 parts by mass or more and 10.0 parts by mass or less.

### <Description of Ultrasound Absorbent>

The hydrosol of the present embodiment may further contain an ultrasound absorbent as another component. The ultrasound absorbent is a material having an action of weakening intensity of ultrasound when the ultrasound is transmitted. A hydrosol having a transmittance of the ultrasound equivalent to that of a living body can be produced by control of transmittance of the ultrasound in the hydrosol.

### <Type of Ultrasound Absorbent>

The ultrasound absorbent which is used in the hydrosol of the present embodiment may be any material as long as a transmission loss of an ultrasound is high, and may be any of a gas, a liquid and a solid. It is preferable for an ultrasound absorbent to be a liquid or a solid from a viewpoint of the dispersion stability in the hydrosol, and is preferable to be a solid from a viewpoint of the particle size and the mechanical stability. Examples of the ultrasound absorbent include urethane rubber, silicone rubber, butadiene rubber, acrylic rubber, nitrile rubber, styrene rubber and latex.

### <Particle Size of Ultrasound Absorbent>

It is preferable that an ultrasound absorbent is homogeneously dispersed in the hydrosol. When a particle size of the ultrasound absorbent is too small, a cohesive force is strong and the ultrasound absorbent is easily aggregated, but when the particle size is too large, sedimentation tends to easily occur. Accordingly, the particle size is preferably 0.1 μm or larger and 50 μm or smaller, and is more preferably 0.2 μm or larger and 30 μm or smaller.

### <Additive Amount of Ultrasound Absorbent>

A content of an ultrasound scattering agent may be appropriately adjusted according to a target scattering effect and is not particularly limited; but when the mass of water is set to 94.0 parts by mass, it is preferable for a content to be 0.1 parts by mass or more and 30.0 parts by mass or less, is more preferable to be 0.5 parts by mass or more and 20.0 parts by mass or less, and is more preferable to be 3.0 parts by mass or more and 10.0 parts by mass or less.

### <Description of Antiseptic Agent>

The hydrosol of the present embodiment may contain an antiseptic agent as another component. In the hydrosol, bacteria and molds generally tend to easily grow proliferously, and accordingly, it is preferable to use the antiseptic agent in order to suppress an influence of deterioration on physical properties.

### <Type of Antiseptic Agent>

The antiseptic agent which can be used is not particularly limited, but it is preferable for the antiseptic agent to be water soluble and have a broad antibacterial spectrum. Examples of the antiseptic agent, a disinfection agent, or an antibacterial agent as a chemical compound which can suppress the growth of bacteria and mold include alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl para-hydroxybenzoate, ethyl para-hydroxybenzoate, propyl para-hydroxybenzoate, butyl para-hydroxybenzoate, oxyquinoline sulfate, phenethyl alcohol and benzyl alcohol.

Among chemical compounds, para-hydroxybenzoic acid esters are desirable because it is desirable to be water soluble, have a broad antibacterial spectrum, and in particular, have little influence on the human body. In addition, methyl para-hydroxybenzoate is particularly preferable from a viewpoint of water solubility.

### <Additive Amount of Antiseptic Agent>

It is preferable that the antiseptic agent is added as appropriate, because an effect varies depending on the respective chemical compounds; but for example, in a case of methyl para-hydroxybenzoate, when a mass of water is set to 94.0 parts by mass, the antiseptic agent may be added in an amount of 0.1 parts by mass or more and 0.3 parts by mass or less; and the effect as an antiseptic agent is sufficient when the antiseptic agent is added in this range.

### <Description of Defoaming Agent>

The hydrosol of the present embodiment may contain a defoaming agent as another component. When air bubbles have entered the hydrosol in producing the hydrosol, ultrasound results in being excessively scattered due to the difference in acoustic impedance between water and the air interface, and accordingly, it is preferable to use the defoaming agent.

### <Type of Defoaming Agent>

Examples of the defoaming agent which can be used include: oils such as mineral oils, fats and fatty oils; surface active agents such as fatty acid, fatty acid ester, phosphate ester and metal soap; and silicone compounds such as silicone oil and dimethylsiloxane. Among the chemical compounds, silicone compounds are particularly preferable which have a high defoaming effect even when the additive amount is small.

### <Additive Amount of Defoaming Agent>

It is preferable that the defoaming agent is added as appropriate, because effects vary according to the respective chemical compounds; but for example, in a case of a silicone compound, when a mass of water is set to 94.000 parts by mass, the defoaming agent may be added in an amount of 0.001 parts by mass or more and 0.100 parts by mass or less, and an effect as a defoaming agent is sufficient when an additive amount is in this range.

### <Description of Thickening Agent>

The hydrosol of the present embodiment may contain a thickening agent as another component. An addition of a thickening agent can enhance a dispersion stability of the ultrasound scattering agent and the ultrasound absorbent in the hydrosol.

Examples of the thickening agent which can be used include: synthetic polymers such as sodium polyacrylate and polyvinyl alcohol; and polysaccharides such as tamarind seed gum, guar gum, succinoglycan, diutan gum, derivatives thereof, and cellulose derivatives. On the other hand, a chemical compound is preferable which does not hinder dissolution and thickening of the cellulose ether by being added.

### <Gelling Agent>

The hydrosol of the present embodiment may contain a gelling agent as another component. Young's modulus of the hydrosol can be finely adjusted by an addition of the gelling agent. Examples of the gelling agent which can be used include: polysaccharides such as agar, carrageenan, pectin, gellan gum, sodium alginate, tamarind seed gum and curdlan; and gelatin.

### <Other Components>

The hydrosol of the present embodiment may contain a coloring agent such as an aqueous dye, and a pH adjusting agent such as a phosphate buffer solution, as other components.

### <Material Having Multiple Functions>

The hydrosol of the present embodiment includes water, cellulose ether, a sound velocity adjusting agent, and other components as necessary, and exhibits a plurality of functions in some cases, depending on a type and amount of the materials to be used. In this case, even if the function is different from the intended function, the material is regarded as being added.

For example, graphite has a high scattering efficiency for ultrasound, and accordingly, functions mainly as an ultrasound scattering agent, but transmits a part of ultrasounds; and also functions as a sound velocity adjusting agent and an ultrasound absorbing material. In this case, even if a purpose is not intended to adjust velocity of sound or transmittance of an ultrasound, the sound velocity adjusting agent or the ultrasound absorbent is regarded to have been added.

### <Components of Hydrosol>

It is preferable that the hydrosol according to the present embodiment includes water, cellulose ether, a sound velocity adjusting agent, and as other components, an ultrasound scattering agent, an antiseptic agent and a defoaming agent. It is preferable for a total mass of these components to be 80 parts by mass or more and 100 parts by mass or less, is more preferable to be 90 parts by mass or more and 100 parts by mass or less, and is further preferable to be 95 parts by mass or more and 100 parts by mass or less, when a mass of the whole hydrosol is set to 100 parts by mass. When the additive amount is within the range, the hydrosol can sufficiently exhibit a function of the present embodiment.

### <Viscosity of Hydrosol>

In dynamic shear viscoelasticity measurement at 25° C. of the hydrosol of the present embodiment, when storage elastic modulus at a frequency of 2.5 Hz is represented by G′_{2.5} and storage elastic modulus at a frequency of 0.25 Hz is represented by G′_{0.25}, it is preferable for G′_{2.5}/G′_{0.25} to be 2.0 or larger and 15 or smaller, and is more preferable to be 2.0 or larger and 10 or smaller. In this way, the hydrosol of the present embodiment may have high viscosity and appropriate elasticity.

### <Method for Analyzing Hydrosol>

Viscoelasticity of the hydrosol according to the present embodiment can be evaluated by measurement using a shear type of dynamic viscoelasticity measuring apparatus. An example of the apparatus is MCR302 available from Anton Paar Japan K.K.

As for measurement conditions for a shear type of dynamic viscoelasticity measurement, it is desirable that measurement strain is 0.01% or larger and 1.00% or smaller, from a viewpoint of measurement error. When the strain is smaller than 0.01%, a stress response from the hydrosol becomes small, and accordingly, a measured value is not reliable. In addition, when the strain is larger than 1.00%, the internal structure may be destroyed due to deformation of the hydrosol, and accordingly, a measured value is less reliable.

In addition, in view of followability of the hydrosol and a measurement tool, it is preferable that a measurement frequency is 0.01 Hz or larger and 10.0 Hz or smaller. When a measured value in a high-frequency side exceeding 10.0 Hz is necessary, a temperature-time conversion rule can be utilized to predict the data in the high-frequency side.

Specifically, a composite curve (master curve) is obtained by performing dynamic viscoelasticity measurement at a plurality of temperatures while changing frequency, and laterally shifting the obtained data for respective temperatures. An amount of lateral shift can be determined by approximation with WLF rule or Arrhenius rule.

### <Method for Discriminating Between Hydrosol and Hydrogel>

It can be determined which state is indicated of the aforementioned hydrosol or hydrogel, with the aforementioned shear type of dynamic viscoelasticity measuring apparatus. Specifically, when a value of tan δ is 1 or larger, which is a ratio of a loss elastic modulus G″ to a storage elastic modulus G′, the state can be determined to indicate a hydrosol, and when the value is smaller than 1, the state can be determined to indicate a hydrogel.

### <Index of Viscosity>

When a viscoelastic body fits Voigt model, storage elastic modulus (G′) increases, as viscosity increases and measurement frequency increases. In other words, a rate of increase in storage elastic modulus G′ between certain frequencies can be used as an index of viscosity.

A viscosity of the hydrosol according to the present embodiment was evaluated by utilization of this characteristic. Specifically, when storage elastic modulus at a frequency of 2.5 Hz was represented by G′_{2.5} and the storage elastic modulus at a frequency of 0.25 Hz was represented by G′_{0.25}, the value of G′_{2.5}/G′_{0.25} which was a ratio between the moduli was determined to have been an index of the viscosity. For information, viscoelasticity was measured under conditions of a strain of 0.1%, a measurement tool of parallel plates with a diameter of 25 mm, and a sample thickness of 0.4 mm.

### <Measurement of Steady-Flow Viscosity>

An index G′_{2.5}/G′_{0.25} of the viscosity of the hydrosol in the present embodiment can be indirectly obtained by measurement of a steady-flow viscosity. The steady-flow viscosity can be measured with a use of an E-type viscometer, etc. A measurement tool of a viscometer may be a parallel plate or a cone plate. As for measurement conditions, it is preferable that a shear rate is 0.1 sec⁻¹ or larger and 1000.0 sec⁻¹ or smaller.

The steady-flow viscosity is a viscosity when a shear force in a same direction is applied, and accordingly is essentially different from that of a dynamic shear viscoelasticity measurement in which a periodic force is applied; and accordingly, it cannot directly provide the index G′_{2.5}/G′_{0.25} of the viscosity.

However, in the hydrosol according to the present embodiment, the Cox-Merz law holds true, in which a relationship between shear rate and steady-flow viscosity in the steady-flow viscosity measurement and a relationship between an angular velocity and a complex viscosity in a dynamic viscoelasticity measurement overlap. In other words, complex viscosity can be indirectly obtained by measurement of the steady-flow viscosity.

Furthermore, the complex viscosity can be converted into a complex elastic modulus (square root of sum of squares of storage elastic modulus and loss elastic modulus), by multiplication by the angular velocity (2×π×frequency).

Here, when it is assumed that tan δ does not change between the frequencies of 0.25 Hz and 2.5 Hz, a ratio G*_{2.5}/G*_{0.25} of complex elastic modulus G*_{2.5} of 2.5 Hz to complex elastic modulus G*_{0.25} Hz of 0.25 Hz can be regarded as equivalent to G′_{2.5}/G′_{0.25} which is a ratio of a storage elastic moduli.

A method for calculating a ratio G′_{2.5}/G′_{0.25} of the storage elastic moduli obtained by the steady-flow viscosity measurement is performed, based on a thought that two presumptions hold true that the Cox-Merz law is satisfied and tan δ does not change between frequencies 0.25 Hz and 2.5 Hz; and accordingly, it is preferable to directly determine the ratio G′_{2.5}/G′_{0.25} of the storage elastic moduli by the shear dynamic viscoelasticity measurement.

### Second Embodiment

The second embodiment is about an ultrasound phantom and an ultrasound phantom set.

The ultrasound phantom of the present embodiment includes: the hydrosol of the present disclosure; and a container that has a retaining portion which is in contact with the hydrosol and retains a shape of the hydrosol, and accommodates the hydrosol.

The ultrasound phantom set of the present embodiment includes: the ultrasound phantom described above; and a second ultrasound phantom which includes a second cellulose ether having an average molecular weight different from the average molecular weight of the cellulose ether contained in the ultrasound phantom, water, a sound velocity adjusting agent, and a second container which accommodates a second cellulose ether, a water, and a sound velocity adjusting agent.

The content will be described below. Some of the substances are as described above, and accordingly, a description thereof will be omitted.

### <Description of Ultrasound Phantom>

The ultrasound phantom in the present disclosure is formed into a portable form which can be used for calibration of an ultrasound diagnostic apparatus by accommodating the aforementioned hydrosol in a container which will be described later. The ultrasound phantom has thus portability, thereby can be distributed by transportation, and can be carried to a vicinity of the ultrasound diagnostic apparatus which a user wants to calibrate.

### <Description of Container>

The container to be used in the ultrasound phantom of the present embodiment is for retaining a shape of the hydrosol of the present embodiment. In other words, in the ultrasound phantom of the present embodiment, the container is a container that has a retaining portion which is in contact with the hydrosol and retains the shape of the hydrosol, and accommodates the hydrosol.

A material of the container is not particularly limited, and may be any material as long as the material does not cause deformation of the ultrasound phantom or exudation of water content. A shape of the container may be a cube, a rectangular parallelepiped, a cylinder, etc., or may be a shape imitating an organ. In addition, the container may be partially filled with a material other than the hydrosol of the present embodiment.

Furthermore, the container may have a lid portion at a position facing an acoustic coupling surface at an upper portion of the container, for a purpose of preventing evaporation of water in the hydrosol. In other words, in the ultrasound phantom of the present embodiment, the container may include a lid portion which faces an acoustic coupling surface, and a main body portion which has a retaining portion for retaining the shape of the hydrosol and is fastened to the lid portion. A material of the lid portion is not particularly limited, but may be any material as long as the material does not cause exudation of water content, and as for the shape, it is preferable to have a structure that adheres to the retaining portion or the main body portion.

### <Acoustic Coupling Surface>

It is preferable that the hydrosol in the ultrasound phantom of the present embodiment has an acoustic coupling surface for acoustically being coupled with an acoustic probe, and that the container has a retaining portion at a position different from the acoustic coupling surface. Furthermore, it is preferable that the hydrosol in the ultrasound phantom of the present embodiment has an acoustic wave reducing portion that reduces a reflection of an acoustic wave emitted from acoustic coupling surface, at a position facing the acoustic coupling surface through the hydrosol.

The acoustic coupling surface in the present embodiment means a surface which is acoustically coupled to the acoustic probe. It is preferable that the acoustic coupling surface is a flat surface, from a viewpoint of transmissibility of an ultrasound. In addition, the acoustic coupling surface may be the hydrosol of the present embodiment, or may be covered with a material other than the hydrosol of the present embodiment. For example, the surface may be covered with a film-like material for a purpose of preventing evaporation.

Here, a sentence that the surface is acoustically coupled to an acoustic probe means that transmission of an ultrasound is satisfactory. An acoustic probe and a human body (or the ultrasound phantom) have a large difference in acoustic impedance, and it is a case that the ultrasound beam results in being reflected and is not efficiently transmitted into the living body.

For this reason, it is necessary to interpose a substance having the acoustic characteristic impedance of the human body between the acoustic probe and the human body; and the acoustic coupling surface which has been interposed can minimize a reflection of the ultrasound at the acoustic coupling surface, and a transmission of the ultrasound becomes satisfactory.

### <Sound Absorbing Material>

The sound absorbing material in the present embodiment is a material that attenuates the ultrasound that has passed through. In other words, the sound absorbing material is a material which is used in the sound reduction portion. Thereby, the ultrasound incident from the acoustic coupling surface is attenuated by passing through the sound absorbing material after having passed through the hydrosol. As a result, the reflected wave from the container can be suppressed.

A material of the sound absorbing material may be any material as long as the transmission loss of the ultrasound is high; and is preferably a rubber material, and is particularly preferably a urethane rubber material. The transmission loss is preferably as high as possible, and is preferably 15 dB·MHz⁻¹·cm⁻¹ or higher. It is more preferable for the transmission loss to be 20 dB·MHz⁻¹·cm⁻¹ or higher, and is further preferable to be 25 dB·MHz⁻¹·cm⁻¹ or higher.

### <Ultrasound Phantom Set>

An ultrasound phantom set can be provided by utilization of the characteristics of the thickening mechanism of the aforementioned cellulose ether. For example, for an ultrasound phantom set having the same storage elastic modulus and different viscosities, a plurality of hydrosols adjusted to have a same storage elastic modulus may be prepared from a plurality of cellulose ethers having different molecular weights. In this way, the ultrasound phantom set can be used for calibration of ultrasound equipment.

In addition, for an ultrasound phantom set having different storage elastic moduli but same viscosity, a plurality of hydrosols adjusted to have a same index of viscosity may be prepared from a plurality of cellulose ethers having different molecular weights.

### Third Embodiment

The third embodiment is about a method for manufacturing the ultrasound phantom.

The method for manufacturing the ultrasound phantom according to the present embodiment includes: producing a mixed liquid in which water, cellulose ether and a sound velocity adjusting agent are mixed; producing a hydrosol by stirring and mixing a mixed liquid; and pouring the hydrosol into a container.

The content will be described below. The respective substances are as described above, and accordingly, the description thereof will be omitted.

The method for manufacturing the ultrasound phantom is not particularly limited. A mixed liquid is produced by mixing water, cellulose ether, a sound velocity adjusting agent, and other components as necessary such as an ultrasound scattering agent, an antiseptic agent and a defoaming agent. By stirring of the mixed liquid, a hydrosol is obtained in which the cellulose ether is at least partially dissolved. In this case, heating or cooling may be performed as necessary.

It is preferable that the method for manufacturing the ultrasound phantom of the present embodiment includes a step of measuring dynamic shear viscoelasticity of the hydrosol after the stirring step, and thereby acquiring a curve of frequency-storage elastic modulus. In this way, a calibration curve of the ultrasound phantom can be obtained.

Here, a distribution of the components contained in the hydrosol includes: a mode in which the components are macroscopically uniformly dispersed in the solvent and are in equilibrium; and a mode in which the components are microscopically uniformly dissolved in the solvent at a molecular level and are in equilibrium. The former includes an ultrasound scattering agent and a defoaming agent, and the latter includes cellulose ether, a sound velocity adjusting agent and an antiseptic agent.

The ultrasound phantom can be obtained by pouring the obtained hydrosol into a container having a desired shape.

According to such a characteristic of a manufacturing method, the materials constituting the ultrasound phantom can be distributed in a packed form, which are adjusted so as to exhibit a desired storage elastic modulus and index of viscosity. A user can produce an ultrasound phantom on-site by mixing a pack of materials into a specified amount of water.

In other words, the method for manufacturing the ultrasound phantom includes: a production step of producing a mixed liquid in which water, cellulose ether and a sound velocity adjusting agent are mixed; a stirring step of producing a hydrosol by stirring and mixing the mixed liquid; and an injection step of pouring the hydrosol into a container having a desired shape. Thereby, an ultrasound phantom can be obtained which contains a hydrosol including water, cellulose ether, and a sound velocity adjusting agent.

It can be confirmed whether or not the hydrosol according to the present embodiment may be produced without any problem, by conducting a step of measuring viscoelasticity of the obtained hydrosol, which is storage elastic modulus at frequencies of 0.25 Hz and 2.5 Hz; and further by obtaining information on whether or not the ratio of the storage elastic moduli at the frequencies of 0.25 Hz and 2.5 Hz is 2.0 or larger and 15 or smaller, before pouring the hydrosol into a container having a desired shape.

The ultrasound phantom according to the present embodiment can be used for calibration of an apparatus as an ultrasound phantom, for example, in an ultrasound diagnostic apparatus such as an ultrasound dispersion method for calculating the viscosity of an organ.

### EXAMPLES

The present disclosure will be described in detail below with reference to Examples, but the present embodiment is not limited to these Examples. In the following formulations, parts are on a mass basis unless otherwise specified.

### <Materials>

Materials used in Examples 1 to 27 and Comparative Examples 1 to 3 are listed below.

### [Water]

Ultrapure water: ultrapure water which is produced by IQ7003 made by Milli-Q Corporation, and is purified by a combination method of a reverse osmosis membrane, continuous ion-exchange, and a sterilizing UV lamp.

### [Cellulose Ether]

C-1 trade name: hi Metolose (registered trade name) hi90SH-4000 (produced by Shin-Etsu Chemical Co., Ltd.)

The above hi Metolose is a hydroxypropyl methyl cellulose which is a cellulose containing β-D-glucose as a main component, and has 1.4 methoxy groups and 0.2 hydroxypropoxy groups per cellulose residue; and has a viscosity of 3,980 mPa·s for a 2 mass % aqueous solution and has a surface treated with glyoxal.
C-2 trade name: hi Metolose (registered trade name) hi90SH-15000 (produced by Shin-Etsu Chemical Co., Ltd.)

The above hi Metolose is a hydroxypropyl methyl cellulose which is a cellulose containing β-D-glucose as a main component and has 1.4 methoxy groups and 0.2 hydroxypropoxy groups per cellulose residue; and has a viscosity of 14,000 mPa·s for a 2 mass % aqueous solution and has a surface treated with glyoxal.
C-3 trade name: hi Metolose (registered trade name) hi90SH-30000 (produced by Shin-Etsu Chemical Co., Ltd.)

The above hi Metolose is a hydroxypropyl methyl cellulose which is a cellulose containing β-D-glucose as a main component and has 1.4 methoxy groups and 0.2 hydroxypropoxy groups per cellulose residue; and has a viscosity of 25,400 mPa·s for a 2 mass % aqueous solution and has a surface treated with glyoxal.
C-4 trade name: hi Metolose (registered trade name) hi90SH-100000 (produced by Shin-Etsu Chemical Co., Ltd.)

The above hi Metolose is a hydroxypropyl methyl cellulose which is a cellulose containing β-D-glucose as a main component and has 1.4 methoxy groups and 0.2 hydroxypropoxy groups per cellulose residue; and has a viscosity of 4,290 mPa·s for a 1 mass % aqueous solution and has a surface treated with glyoxal.
C-5 trade name: Metolose (registered trade name) 60SH-4000 (produced by Shin-Etsu Chemical Co., Ltd.)

The above Metolose is a hydroxypropyl methyl cellulose which is a cellulose containing β-D-glucose as a main component and has 1.9 methoxy groups and 0.3 hydroxypropoxy groups per cellulose residue; and has a viscosity of 4,000 mPa·s for a 2 mass % aqueous solution and has a surface untreated with glyoxal.
C-6 trade name: Metolose (registered trade name) SEB-4000 (produced by Shin-Etsu Chemical Co., Ltd.)

The above Metolose is a hydroxyethyl methyl cellulose which is a cellulose containing β-D-glucose as a main component and has 1.5 methoxy groups and 0.2 hydroxyethoxy groups per cellulose residue; and has a viscosity of 4,000 mPa·s for a 2 mass % aqueous solution and has a surface untreated with glyoxal.
C-7 trade name: Metolose (registered trade name) SM-4000 (produced by Shin-Etsu Chemical Co., Ltd.)

The above Metolose is a methyl cellulose which is a cellulose containing β-D-glucose as a main component and has 1.8 methoxy groups per cellulose residue; and has a viscosity of 4,000 mPa·s for a 2 mass % aqueous solution and has a surface untreated with glyoxal.

### [Sound Velocity Adjusting Agent]

Urea (Kishida Chemical Co., Ltd.)
Glycerin (Kishida Chemical Co., Ltd.)

### [Other Components]

Other polysaccharides: agar (Kishida Chemical Co., Ltd.)

Thickening agent: glycerin (Kishida Chemical Co., Ltd.), which is a water-soluble liquid and accordingly affects a velocity of sound.

Ultrasound scattering agent: graphite, trade name: Nicabeads (registered trade name) ICB1020 (Nippon Carbon Co., Ltd.)
Scaly graphite 1: trade name: MCP-10 (Nippon Graphite Industries Co., Ltd.), with average particle size of 10 μm
Scaly graphite 2: trade name: MCP-15 (Nippon Graphite Industries Co., Ltd.), with average particle size of 15 μm

Antiseptic agent: methyl para-hydroxybenzoate (Kishida Chemical Co., Ltd.)

Defoaming agent: silicone-based defoaming agent, trade name: KS-537 (Shin-Etsu Chemical Co., Ltd.)

### <Evaluation Method>

### [Ratio of Storage Elastic Moduli]

A hydrosol which has been obtained by stirring a mixed liquid in which water, cellulose ether, a sound velocity adjusting agent, and as necessary, other components such as an ultrasound scattering agent, an antiseptic agent and a defoaming agent are mixed was subjected to measurement of dynamic viscoelasticity with an use of a viscoelasticity measuring apparatus (MCR302 manufactured by Anton Paar Japan K.K.) and a parallel plate with a diameter of 25 mm, at a temperature of 25° C. and two types of frequencies of 0.25 Hz and 2.5 Hz, and the storage elastic modulus was obtained at each of the frequencies.

When a storage elastic modulus at a frequency of 2.5 Hz was represented by G′_{2.5} and storage elastic modulus at a frequency of 0.25 Hz was represented by G′_{0.25}, in a case where G′_{2.5}/G′_{0.25} was 2.0 or larger and 15 or smaller, the evaluation was determined to be OK.

When the G′_{2.5}/G′_{0.25} is 2.0 or larger, the hydrosol can be used as an ultrasound phantom having high viscosity. On the other hand, when the G′_{2.5}/G′_{0.25} has exceeded 15, the hydrosol is in such a state that there is almost no elasticity, and accordingly, is not suitable as the ultrasound phantom.

### [Measurement of Velocity of Sound]

In order to measure the velocity of sound, a sample for sound velocity measurement was produced by filling a tool for the sound velocity measurement having a diameter of 64 mm and a length of 40 mm, with the hydrosol, and by sealing both ends with acrylic plates.

A specific method of measuring the velocity of sound is as follows. Fill a water tank with water and record a water temperature. A transducer (V328-SU manufactured by Olympus Corporation, transmission frequency of 3.5 MHz) and a needle-type hydrophone (manufactured by Toray Engineering D Solutions Co. Ltd.) are installed in the water, and a tool filled with water for the sound velocity measurement is installed therebetween.

In this state, the ultrasound transmitted from the transducer is received by the hydrophone, and data of a waveform with respect to time is obtained. Next, a sample for a sound velocity measurement, which has been filled with hydrosol, is installed between the transducer and the hydrophone, and data of a waveform with respect to time is obtained in a same way.

The waveform when only water is filled and the waveform when the hydrosol is installed are subjected to a cross-correlation analysis, and a delay time of an arrival time of the waveform is measured.

When the delay time is represented by τ, a thickness of the sample is represented by t, and the water temperature is represented by T, velocity of sound C₁ of water and velocity of sound C₂ of the sample can be obtained according to the following expressions.

$${C}_{1} = 1403 + 5 \times T - 0.06 \times {T}^{2} + 0.0003 \times {T}^{3}$$

$${C}_{2} = t / ( T + t / {C}_{1} )$$

### Examples 1 to 40, and Comparative Examples 1 to 3

### <Method for Producing Ultrasound Phantom>

### MANUFACTURE EXAMPLES

As an example of a manufacturing procedure of the ultrasound phantom, a production procedure of Example 1 will be described below. In the other Examples 2 to 40 and Comparative Examples 1 to 3, the ultrasound phantoms were produced, by changing only types and amounts of materials according to compositions in Table 1 and Table 2, without changing a production procedure of Example 1.

An ultrasound absorbing tile (AptflexF28 manufactured by Precision Acoustics, transmission loss of 30 dB·MHz⁻¹·cm⁻¹) which was cut into a width of 100 mm, a depth of 100 mm, and a height of 10 mm was attached to a bottom portion of a container which is formed from polypropylene, has an inside diameter having a width of 120 mm and a depth of 120 mm, and has a height of 90 mm, with a water-resistant double-sided tape.

Ultrapure water in an amount of 1410 g and a defoaming agent (KS-537) in an amount of 0.03 g were put into a 2 L disposable cup, and a stirring apparatus was installed which was equipped with a flat-panel type stirring blade with a diameter of 100 mm. A mixture of 90 g of a sound velocity adjusting agent (urea), 60 g of an ultrasound scattering agent (Nicabeads ICB1020), 0.75 g of an antiseptic agent (methyl para-hydroxybenzoate), and 37.5 g of cellulose ether C-1 (hi Metolose hi90SH-4000) was sufficiently mixed, was then added little by little while the resultant was stirred at 150 rpm, and was stirred at room temperature for 1 hour at 150 rpm. Next, rotational velocity was lowered to 75 rpm and stirring was performed for 30 minutes to produce a hydrosol.

Next, the above hydrosol was poured into a container which is formed from polypropylene and is equipped with the above ultrasound absorbing tile, from an upper end of a container to a height of 1 cm to produce an ultrasound phantom.

The obtained hydrosol was used to evaluate the above index of viscosity, and it was checked whether a hydrosol having a desired viscosity was obtained. Furthermore, a tool for the sound velocity measurement was filled with the obtained hydrosol, and both ends were sealed with acrylic plates to produce a sample for sound velocity measurement and the velocity of sound was measured according to the above method.

As an example of a manufacturing procedure of the ultrasound phantom, a production procedure of Comparative Example 1 will be described. In Comparative Example 2, an ultrasound phantom was produced by changing only a type and amount of materials according to the composition in Table 1, without changing the procedure.

Ultrapure water in an amount of 1410 g and a defoaming agent (KS-537) in an amount of 0.03 g were put into a 2 L separable flask, and a stirring apparatus was installed which was equipped with a stirring blade having a half-moon shape of 75 mm. A mixture of 90 g of a sound velocity adjusting agent (urea), 60 g of an ultrasound scattering agent (Nicabeads (registered trade mark) ICB1020), 0.75 g of an antiseptic agent (methyl para-hydroxybenzoate), and 17.5 g of agar was sufficiently mixed, was then added little by little while the resultant was stirred at 150 rpm, and was stirred for 1 hour at 150 rpm in an oil bath at 90° C. to produce a sol liquid in which the agar was dissolved.

For information, components other than water, which are described in the embodiments and Examples described in the present specification, are weighed while the mass is regarded as a mass of a sufficiently dried anhydride (dry mass). The dry mass can be determined by a loss on drying method specified in JIS K0068.

After the evaluation for Table 1, the above mixed solution was poured into a container which is formed from polypropylene and is equipped with the above ultrasound absorbing tile, from an upper end of the container to a height of 1 cm, and an ultrasound phantom was produced.

The tool for sound velocity measurement was filled with the obtained hydrosol, and both ends were sealed with acrylic plates. The tool was left at room temperature overnight, thereby a sample for sound velocity measurement was produced, and the velocity of sound was measured according to the above method.

In addition, the obtained hydrosol was cooled to room temperature, and was used to evaluate the index of viscosity.

The obtained results are shown in Table 1 and Table 2.

TABLE 1

| | Formulation [parts by mass] | | | | | | | | | | |
|---|---|---|---|---|---|---|---|---|---|---|---|
| | | | | | | | | | | Defoaming | Methyl |
| | Water | C-1 | C-2 | C-3 | C-4 | Agar | Urea | Glycerin | Graphite | agent | paraben |
| Example 1 | 94.0 | 2.5 | | | | | 6.0 | | 4.0 | 0.02 | 0.05 |
| Example 2 | 94.0 | 3.7 | | | | | 6.0 | | 4.0 | 0.02 | 0.05 |
| Example 3 | 94.0 | 4.0 | | | | | 6.0 | | | | 0.05 |
| Example 4 | 94.0 | 4.1 | | | | | 6.0 | | 4.0 | 0.02 | 0.05 |
| Example 5 | 94.0 | 4.3 | | | | | 6.0 | | 4.0 | 0.02 | 0.05 |
| Example 6 | 94.0 | 4.4 | | | | | 6.0 | | 4.0 | 0.02 | 0.05 |
| Example 7 | 94.0 | 5.5 | | | | | 6.0 | | | | 0.05 |
| Example 8 | 94.0 | | 1.5 | | | | 6.0 | | | | 0.05 |
| Example 9 | 94.0 | | 3.0 | | | | 6.0 | | | | 0.05 |
| Example 10 | 94.0 | | 3.1 | | | | 6.0 | | 4.0 | 0.02 | 0.05 |
| Example 11 | 94.0 | | 3.4 | | | | 6.0 | | 4.0 | 0.02 | 0.05 |
| Example 12 | 94.0 | | 3.8 | | | | 6.0 | | 4.0 | 0.02 | 0.05 |
| Example 13 | 94.0 | | 4.5 | | | | 6.0 | | | | 0.05 |
| Example 14 | 94.0 | | | 1.5 | | | 6.0 | | | | 0.05 |
| Example 15 | 94.0 | | | 2.5 | | | 6.0 | | | | 0.05 |
| Example 16 | 94.0 | | | 3.1 | | | 6.0 | | 4.0 | 0.02 | 0.05 |
| Example 17 | 94.0 | | | 3.4 | | | 6.0 | | 4.0 | 0.02 | 0.05 |
| Example 18 | 94.0 | | | 3.5 | | | 6.0 | | | | 0.05 |
| Example 19 | 94.0 | | | 3.8 | | | 6.0 | | 4.0 | 0.02 | 0.05 |
| Example 20 | 94.0 | | | | 1.0 | | 6.0 | | | | 0.05 |
| Example 21 | 94.0 | | | | 2.0 | | 6.0 | | | | 0.05 |
| Example 22 | 94.0 | | | | 2.9 | | 6.0 | | 4.0 | 0.02 | 0.05 |
| Example 23 | 94.0 | | | | 3.0 | | 6.0 | | | | 0.05 |
| Example 24 | 94.0 | | | | 3.1 | | 6.0 | | 4.0 | 0.02 | 0.05 |
| Example 25 | 94.0 | | | | 3.3 | | 6.0 | | 4.0 | 0.02 | 0.05 |
| Example 26 | 94.0 | | | | 3.4 | | 6.0 | | 4.0 | 0.02 | 0.05 |
| Example 27 | 94.0 | | | | 3.4 | | | 9.0 | | | |
| Comparative | 91.0 | | | | | 1.5 | | 9.0 | 4.0 | | 0.05 |
| Example 1 | | | | | | | | | | | |
| Comparative | 94.0 | | | | | 1.5 | 6.0 | | 4.0 | | 0.05 |
| Example 2 | | | | | | | | | | | |
| Comparative | 94.0 | | | | 3.4 | | | | | | |
| Example 3 | | | | | | | | | | | |

Evaluation item

| | G′_{2.5}/G′_{0.25} | Velocity of sound [m/s] |
|---|---|---|
| Example 1 | 7.1 | 1537 |
| Example 2 | 8.5 | 1537 |
| Example 3 | 7.1 | 1540 |
| Example 4 | 8.0 | 1536 |
| Example 5 | 7.7 | 1539 |
| Example 6 | 7.4 | 1538 |
| Example 7 | 5.5 | 1540 |
| Example 8 | 5.0 | 1536 |
| Example 9 | 4.3 | 1539 |
| Example 10 | 4.7 | 1537 |
| Example 11 | 4.2 | 1537 |
| Example 12 | 4.1 | 1536 |
| Example 13 | 3.5 | 1538 |
| Example 14 | 5.5 | 1537 |
| Example 15 | 4.6 | 1535 |
| Example 16 | 4.2 | 1537 |
| Example 17 | 4.1 | 1538 |
| Example 18 | 3.6 | 1539 |
| Example 19 | 3.7 | 1538 |
| Example 20 | 5.8 | 1539 |
| Example 21 | 3.6 | 1538 |
| Example 22 | 3.5 | 1535 |
| Example 23 | 3.1 | 1535 |
| Example 24 | 3.3 | 1539 |
| Example 25 | 3.2 | 1538 |
| Example 26 | 3.2 | 1537 |
| Example 27 | 3.0 | 1537 |
| Comparative Example 1 | 1.1 | 1537 |
| Comparative Example 2 | 1.0 | 1539 |
| Comparative Example 3 | 3.1 | 1490 |

TABLE 2

| | Formulation [parts by mass] | | | | | | | |
|---|---|---|---|---|---|---|---|---|
| | Water | C-1 | C-4 | C-5 | C-6 | C-7 | Urea | Graphite |
| Example 28 | 94.0 | 4.3 | | | | | 6.0 | |
| Example 29 | 94.0 | 4.3 | | | | | 6.0 | |
| Example 30 | 94.0 | | 3.4 | | | | 6.0 | |
| Example 31 | 94.0 | | 3.4 | | | | 6.0 | |
| Example 32 | 94.0 | | | 2.5 | | | 6.0 | 4.0 |
| Example 33 | 94.0 | | | 4.0 | | | 6.0 | 4.0 |
| Example 34 | 94.0 | | | 5.5 | | | 6.0 | 4.0 |
| Example 35 | 94.0 | | | | 2.5 | | 6.0 | 4.0 |
| Example 36 | 94.0 | | | | 4.0 | | 6.0 | 4.0 |
| Example 37 | 94.0 | | | | 5.5 | | 6.0 | 4.0 |
| Example 38 | 94.0 | | | | | 2.5 | 6.0 | 4.0 |
| Example 39 | 94.0 | | | | | 4.0 | 6.0 | 4.0 |
| Example 40 | 94.0 | | | | | 5.5 | 6.0 | 4.0 |

| | Formulation [parts by mass] | | | | Evaluation item | |
|---|---|---|---|---|---|---|
| | Scaly | Scaly | Defoaming | Methyl | | Velocity of |
| | graphite 1 | graphite 2 | agent | paraben | G′_{2.5}/G′_{0.25} | sound [m/s] |
| Example 28 | 7.0 | | 0.02 | 0.05 | 7.7 | 1536 |
| Example 29 | | 7.0 | 0.02 | 0.05 | 7.8 | 1536 |
| Example 30 | 6.3 | | 0.02 | 0.05 | 3.2 | 1534 |
| Example 31 | | 6.3 | 0.02 | 0.05 | 3.2 | 1535 |
| Example 32 | | | 0.02 | 0.05 | 8.0 | 1535 |
| Example 33 | | | 0.02 | 0.05 | 7.6 | 1537 |
| Example 34 | | | 0.02 | 0.05 | 7.4 | 1533 |
| Example 35 | | | 0.02 | 0.05 | 8.1 | 1536 |
| Example 36 | | | 0.02 | 0.05 | 7.6 | 1534 |
| Example 37 | | | 0.02 | 0.05 | 7.2 | 1536 |
| Example 38 | | | 0.02 | 0.05 | 7.6 | 1538 |
| Example 39 | | | 0.02 | 0.05 | 7.2 | 1535 |
| Example 40 | | | 0.02 | 0.05 | 6.3 | 1536 |

In Table 1 and Table 2, numerical values of the respective materials in a column of the formulation indicate the mass (where unit is parts by mass) of the respective materials.

From the results of Table 1 and Table 2, in all of Examples 1 to 40 according to the present disclosure, G′_{2.5}/G′_{0.25} was in a range of 2.0 to 15.0, and the velocity of sound was in a range of 1530 to 1540 m/s. In other words, it has been confirmed that both of the high viscosity and a temperature close to that of a living body are achieved.

In the results of Table 1, Comparative Example 1 which did not use cellulose ether showed the G′_{2.5}/G′_{0.25} of smaller than 2.0 and showed insufficient viscosity. In addition, in Comparative Example 2 that used glycerin which is a thickening agent having an effect of adjusting the velocity of sound, when the velocity of sound was adjusted to 1530 to 1540 m/s which is a region of a living body, G′_{2.5}/G′_{0.25} was smaller than 2.0, and the viscosity could not be sufficiently increased.

In addition, in Comparative Example 3 to which the sound velocity adjusting agent was not added, the viscosity exceeded an evaluation criterion, but the velocity of sound could not reach the region of a living body.

The present disclosure can provide: a hydrosol that can control the viscosity (having high viscosity) and is applied to an ultrasound phantom in which a velocity of sound is close to that in a living body; an ultrasound phantom using the hydrosol; and a method for manufacturing the same. The ultrasound phantom and the ultrasound phantom set of the present disclosure can be used for calibration of an ultrasound diagnostic apparatus.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Applications No. 2024-051949, filed Mar. 27, 2024 and No 2025-022738, filed Feb. 14, 2025, which are hereby incorporated by reference herein in their entirety.

What is claimed is:

1. A hydrosol to be applied to an ultrasound phantom, comprising:
water, cellulose ether, and a sound velocity adjusting agent.

2. The hydrosol according to claim 1, wherein the cellulose ether has a hydroxy group derived from a glucose residue substituted with at least one selected from the group consisting of a methoxy group, a hydroxyethoxy group and a hydroxypropoxy group.

3. The hydrosol according to claim 1, wherein one glucose residue of the cellulose ether is modified with a methoxy group at a proportion of 0.4 or larger and 2.4 or smaller, with a hydroxyethoxy group at a proportion of 0 or larger and 0.6 or smaller, and with a hydroxypropoxy group at a proportion of 0 or larger and 0.5 or smaller, on average.

4. The hydrosol according to claim 1, wherein the cellulose ether is surface-treated with glyoxal.

5. The hydrosol according to claim 1, wherein in dynamic shear viscoelasticity measurement at 25° C., when a storage elastic modulus at a frequency of 2.5 Hz is represented by G′_{2.5} and a storage elastic modulus at a frequency of 0.25 Hz is represented by G′_{0.25}, G′_{2.5}/G′_{0.25} is 2.0 or larger and 15 or smaller.

6. The hydrosol according to claim 1, wherein in dynamic shear viscoelasticity measurement at 25° C., when a storage elastic modulus at a frequency of 2.5 Hz is represented by G′_{2.5} and a storage elastic modulus at a frequency of 0.25 Hz is represented by G′_{0.25}, G′_{2.5}/G′_{0.25} is 2.0 or larger and 10 or smaller.

7. An ultrasound phantom comprising:
the hydrosol according to claim 1; and
a container which has a retaining portion that is in contact with the hydrosol and retains a shape of the hydrosol, and accommodates the hydrosol.

8. The ultrasound phantom according to claim 7, wherein the hydrosol has an acoustic coupling surface for acoustically being coupled with an acoustic probe, and
the container has the retaining portion at a position different from the acoustic coupling surface.

9. The ultrasound phantom according to claim 8, further comprising an acoustic wave reducing portion that reduces a reflection of an acoustic wave emitted from the acoustic coupling surface, at a position facing the acoustic coupling surface through the hydrosol.

10. The ultrasound phantom according to claim 8, wherein the container comprises: a lid portion facing the acoustic coupling surface; the retaining portion; and a main body portion fastened to the lid portion.

11. An ultrasound phantom set comprising:
the ultrasound phantom according to claim 7; and
a second ultrasound phantom that comprises a second cellulose ether having an average molecular weight different from the average molecular weight of the cellulose ether, water, a sound velocity adjusting agent, and a second container that accommodates the second cellulose ether, the water, and the sound velocity adjusting agent.

12. A method for manufacturing an ultrasound phantom comprising:
producing a mixed liquid in which water, cellulose ether and a sound velocity adjusting agent are mixed;
producing a hydrosol by stirring and mixing the mixed liquid; and
pouring the hydrosol into a container.

13. The method for manufacturing the ultrasound phantom according to claim 12, further comprising: measuring a dynamic shear viscoelasticity of the hydrosol after the stirring, and thereby acquiring a curve of frequency-storage elastic modulus.
